(19) United States
(12) Patent Application Publication
(10) Pub. No.: US 2020/0214943 A1
(43) Pub. Date: Jul. 9, 2020

(54) NANOPARTICLE-CONTAINING COMPOSITION FOR HIGHLY EFFICIENT SKIN BRIGHTENING, WHITENING, MOISTURIZING AND SPOT FADING, AND PREPARATION METHOD THEREOF

(71) Applicant: Hong-Mei DAI, Taichung City (TW)

(72) Inventor: Wen-Lie CHANG, New Taipei City (TW)

(73) Assignee: Hong-Mei DAI, Taichung City (TW)

(21) Appl. No.: 16/713,086

(22) Filed: Dec. 13, 2019

(30) Foreign Application Priority Data
Jan. 9, 2019 (TW) 108100868

(51) Int. Cl. A61K 8/02; A61K 8/19; A61K 8/27; A61K 8/44; A61K 8/65; A61K 8/04; A61Q 19/02; A61Q 19/00; A61Q 17/04; B82Y 5/00

(52) U.S. Cl. CPC A61K 8/0204; A61K 8/19; A61K 8/27; A61K 8/44; A61K 8/65; A61K 8/042; A61K 2800/5922; A61Q 19/007; A61Q 17/04; B82Y 5/00; A61K 2800/81; A61K 2800/413; A61Q 19/02

(57) ABSTRACT

A nanoparticle-containing composition for highly efficient skin brightening, whitening, moisturizing is prepared by mixing noble metal, such as platinum, gold, palladium and silver, nanoparticles, zinc oxide nanoparticles, tranexamic acid powder, a dispersant, deionized water and gelatin or a cream in predetermined weight percentages. An aqueous solution of the noble metal nanoparticles is mixed with an aqueous solution of the zinc oxide nanoparticles to produce a primary mixed solution, whose clear upper-layer liquid is extracted and added with the deionized water to produce a secondary mixed solution. The secondary mixed solution is added with the tranexamic acid powder and the dispersant to produce a final mixed solution, whose clear upper-layer liquid is extracted, added with the gelatin or cream and thoroughly stirred until the desired consistency is reached.

### FIELD OF THE INVENTION

The present disclosure relates to a nanoparticle-containing composition for highly efficient skin brightening, whitening, moisturizing and spot fading. More particularly, the present disclosure relates to a composition that takes the form of a gel or cream and can be evenly applied over and adhere to a user's skin in order for the ingredients of the nanoparticle-containing composition such as platinum nanoparticles, gold nanoparticles, palladium nanoparticles, silver nanoparticles, zinc oxide nanoparticles and tranexamic acid to produce long-lasting health-maintaining and cosmetic effects, including antibacterial, skin brightening, whitening, moisturizing and spot fading effects, on the skin.

### BACKGROUND OF THE INVENTION

Global statistical studies have shown that city dwellers spend about 80%˜90% of each day living or working in a closed indoor environment (e.g., home, office, factory or other closed buildings). Particular attention, therefore, should be paid to how people's physical health, including skin health, is affected by the dry air and the dirt, bacteria, viruses, mold and other airborne pollutants in such closed spaces. In addition to skin damage resulting from prolonged exposure to the dry and dirty air of a closed air conditioning system, those who live and work in a tropical or subtropical region must bear the harm inflicted by outdoor ultraviolet (UV) radiation on the skin. Thus, given the detrimental indoor and outdoor environments, plus the busy and stressful urban life, it seems inevitable for metropolitans to have premature skin aging problems, dark spots on their skin, pimple scars or other skin issues. To prevent the aforesaid dermatological conditions, city residents must clean and take care of their skin on a regular basis. Such skin care procedures are especially important to and should be thoroughly practiced by urban dwellers who pursue physical attractiveness.

In light of the above, the cosmetics industry has brought out a variety of gel- or cream-like products dedicated to skin care. These products, however, serve mainly to “moisturize” the skin and fail to provide other health-maintaining or cosmetic effects, such as an “antibacterial”, “skin brightening”, “skin whitening” or “spot fading” effect. It is an important issue for cosmetics manufacturers to design a novel cosmetic and skin care product that is targeted at those who live and work in an air-conditioned environment and stay tense and busy for long hours every day. It is desirable that this novel cosmetic and skin care product can provide “highly efficient moisturization” over a long time; can help the user's skin to absorb moisture to raise a water content of the skin and to protect the skin from the dryness of an air-conditioned environment; can keep the user's skin delicate, supple and radiant; and can additionally produce a “highly efficient antibacterial effect”, a “highly efficient skin brightening and whitening effect” and a “highly efficient spot fading effect”. Preferably, the “highly efficient antibacterial effect” of this product is long-lasting, takes place regardless of the presence or absence of light and can degrade the bacteria, viruses, mold or other organic substances on the user's skin to effectively ward off and eliminate germs, to ensure the safety and health of the user's skin, to effectively reduce the risk of leaving pimple scars on the face and to effectively “lighten” and “whiten” the spots or other blemishes caused by exposure to UV radiation. The present disclosure aims to address the very issue of designing a novel cosmetic and skin care product that has the foregoing desirable properties.

### BRIEF SUMMARY OF THE INVENTION

In view of the afore-referenced inadequacies of the conventional skin care products, the inventor of the present invention conducted an extensive research and repeated experiments and finally succeeded in developing a nanoparticle-containing composition for highly efficient skin brightening, whitening, moisturizing and spot fading. The nanoparticle-containing composition can be easily and evenly applied over and adhere to a user's skin, in order for the ingredients of the nanoparticle-containing composition (such as platinum nanoparticles, gold nanoparticles, palladium nanoparticles, silver nanoparticles, zinc oxide nanoparticles and tranexamic acid, which are evenly mixed and dispersed in the nanoparticle-containing composition) to produce long-lasting health-maintaining and cosmetic effects, including antibacterial, skin brightening, whitening, moisturizing and spot fading effects, on the skin.

One objective of the present disclosure is to provide a nanoparticle-containing composition for highly efficient skin brightening, whitening, moisturizing and spot fading. The nanoparticle-containing composition is prepared by mixing ingredients including platinum nanoparticles, gold nanoparticles, palladium nanoparticles, silver nanoparticles, zinc oxide nanoparticles, tranexamic acid powder, a dispersant, deionized water and gelatin or a cream in predetermined weight percentages. The platinum nanoparticles make up 5 ppm˜30 ppm by weight of the nanoparticle-containing composition, are evenly dispersed in the nanoparticle-containing composition and can neutralize free radicals, help a user's skin to absorb moisture to raise a water content of the skin, and keep the skin delicate, supple and radiant. The gold nanoparticles make up 3 ppm˜15 ppm by weight of the nanoparticle-containing composition; are evenly dispersed in the nanoparticle-containing composition; can stimulate the proliferation of, and the autosynthesis of collagen by, fibroblasts to invigorate the skin; and due to their outstanding antioxidation ability, can facilitate skin absorption of the other beneficial ingredients of the nanoparticle-containing composition so as to lighten the spots or other blemishes on the skin surface. The palladium nanoparticles make up 8 ppm˜32 ppm by weight of the nanoparticle-containing composition, are evenly dispersed in the nanoparticle-containing composition, are an excellent catalyst capable of rapidly absorbing the hydrogen in water in a large amount and can work with the gold nanoparticles such that the hydrogen absorbed by the palladium nanoparticles and the oxygen absorbed by the gold nanoparticles gradually combine into a hydrogen peroxide-like structure that can decompose the cell pigments in the epidermis of the skin and thus lighten the spots on the skin persistently without injuring the skin. The silver nanoparticles make up 120 ppm˜480 ppm by weight of the nanoparticle-containing composition, have particle sizes smaller than 10 nm, are evenly dispersed in the nanoparticle-containing composition, are totally harmless to the human body and can effectively inhibit more than 600 species or strains of pathogenic bacteria, thereby preventing skin infection by those pathogenic bacteria and ensuring skin health effectively. The zinc oxide nanoparticles make up 200 ppm˜1000 ppm by weight of the nanoparticle-containing composition, are evenly dispersed in the nanoparticle-containing composition, can provide comprehensive skin protection by reflecting and scattering UV radiation and can effectively prevent the skin from aging and having a dark and dull color after exposure to UV radiation. The tranexamic acid—which can be in the form of water-soluble white crystalline powder, is clinically used as an “antihemorrhagic agent” or “anticoagulant” and has been officially approved by the Ministry of Health and Welfare of Taiwan as a skin whitening and beautifying ingredient—makes up 1%˜5% by weight of the nanoparticle-containing composition. The deionized water makes up 70%˜80% by weight of the nanoparticle-containing composition and is used to blend the aforesaid ingredients together. The dispersant makes up 1%˜5% by weight of the nanoparticle-containing composition and is used in order for the aforesaid ingredients, except for the deionized water, to be evenly mixed and dispersed in the deionized water. The gelatin or cream makes up 0.5%˜10% by weight of the nanoparticle-containing composition and serves as the gelation agent in the nanoparticle-containing composition in order for the foregoing ingredients to be evenly mixed and dispersed in the nanoparticle-containing composition and be evenly applied over and adhere to the user's skin so as to produce long-lasting health-maintaining and cosmetic effects, including antibacterial, skin brightening, whitening, moisturizing and spot fading effects.

Another objective of the present disclosure is to provide a preparation method of the aforesaid nanoparticle-containing composition for highly efficient skin brightening, whitening, moisturizing and spot fading. The preparation method is carried out as follows. To begin with, an aqueous solution of the noble metal nanoparticles, including the platinum nanoparticles, the gold nanoparticles, the palladium nanoparticles and the silver nanoparticles, is mixed with an aqueous solution of the zinc oxide nanoparticles, and the mixture is stirred for at least 1 hour to produce a primary mixed solution in which the noble metal nanoparticles and the zinc oxide nanoparticles in the original aqueous solutions are sufficiently and evenly mixed and dispersed. The primary mixed solution is allowed to rest for at least 12 hours, before the clear upper-layer liquid of the primary mixed solution is taken out. The clear upper-layer liquid is then added with the deionized water to produce a secondary mixed solution, which in turn is added with the tranexamic acid powder and the dispersant to produce a final mixed solution. The final mixed solution is heated to 40˜60° C. and stirred for at least 1 hour until the tranexamic acid powder is dissolved and is evenly mixed and dispersed in the final mixed solution. After that, the final mixed solution is cooled down to room temperature (e.g., 25° C.), and the clear upper-layer liquid of the cooled final mixed solution is taken out as the base mixed solution of the nanoparticle-containing composition. The base mixed solution is added with the gelatin (or cream) and is heated to 40˜60° C. and stirred for at least 1 hour, wherein the gelatin (or cream) makes up 0.5%˜3% (or 5%˜10%) by weight of the nanoparticle-containing composition. Once the base mixed solution and the gelatin (or cream) are sufficiently and evenly mixed and have the desired gel-like (or cream-like) consistency, the nanoparticle-containing composition for highly efficient skin brightening, whitening, moisturizing and spot fading is completed. The nanoparticle-containing composition can be easily and evenly applied over and adhere to a user's skin, in order for the platinum nanoparticles, the gold nanoparticles, the palladium nanoparticles, the silver nanoparticles, the zinc oxide nanoparticles and the tranexamic acid, all of which are now evenly mixed and dispersed in the nanoparticle-containing composition, to produce long-lasting health-maintaining and cosmetic effects, including antibacterial, skin brightening, whitening, moisturizing and spot fading effects.

### BRIEF DESCRIPTION OF THE SEVERAL VIEWS OF THE DRAWINGS

The objectives, technical features and effects of the present invention can be better understood by referring to the following detailed description of some illustrative embodiments in conjunction with the accompanying drawing, in which:

FIG. 1 is a flowchart of the preparation method according to the present disclosure.

### DETAILED DESCRIPTION OF THE INVENTION

A “catalyst” is an additive for reducing the energy required for a chemical reaction to take place between the substances to which the catalyst is applied, and for accelerating the chemical reaction without reacting with, and thus damaging, the structures of those substances. A “photocatalyst” is a catalyst that utilizes the optical energy in nature to produce a catalyzing effect. Many nanoscale photocatalysts can excite ambient oxygen and water molecules when excited by light, turning those molecules into highly oxidizing free radicals such as H₂· and O₂·. These free radicals can decompose organic and inorganic substances that are harmful to the human body or the environment, and the decomposition reactions involved are rapid and pollution-free. Furthermore, a photocatalyst typically has a semiconductor crystal structure. When this semiconductor crystal structure is hit by UV photons or other radiations (e.g., α rays, β rays, γ rays or X rays), an electron is driven out of the structure, leaving a highly oxidizing and positively charged electron hole, wherein the electron and the hole are chemically referred to as an “electron-hole pair”. When the electron meets an oxygen molecule (O₂) in the air, a highly reactive superoxide radical (·O₂) is formed. When the electron hole meets a water molecule (H₂O) in the air, a photochemical reaction occurs in which the electron hole captures the unpaired electron of the hydroxyl group of the water molecule, and the hydroxyl group without the unpaired electron becomes an unstable hydroxyl radical (·OH). To stabilize itself, the unstable hydroxyl radical tends to capture an electron from whatever organic substance it meets, whether the organic substance is outside the photocatalyst or attached to the surface of the photocatalyst. The affected organic substance will be oxidized as a result and turned into water and carbon dioxide, which dissipate in the air.

Zinc oxide nanoparticles has great deodorizing and antibacterial effects and have been added to everyday products as well as medicines to treat various dermatological conditions. For example, zinc oxide nanoparticles are added to antibacterial ointment to inhibit the growth of bacteria; to baby powder, diaper rash ointment and calamine cream to deal with diaper rash; and to shampoo to fight dandruff. Zinc oxide nanoparticles are also the main ingredient of the so-called zinc oxide tape, which is used by athletes as a bandage to prevent soft tissue injuries. In addition, research results have shown that zinc oxide can reflect UVA (i.e., radiation with wavelengths ranging from 320 to 400 nm) and UVB (i.e., radiation with wavelengths ranging from 280 to 320 nm) over the widest ranges and provide comprehensive UVA and UVB blockage stably. The U.S. Food and Drug Administration, therefore, has approved the use of zinc oxide as an active ingredient in sunscreen oil, sunscreen lotion and sunscreen cream. Besides, zinc oxide will not be absorbed by the human body when left on the skin and can hence protect the skin effectively against UV radiation without causing skin irritations, skin allergies or acnes.

The effects of the noble metal nanoparticles including the platinum nanoparticles, the gold nanoparticles, the palladium nanoparticles and the silver nanoparticles are as follows. Silver, when rendered into nanoparticles, has small particle sizes and consequently a large surface area as a whole, which leads to high activity, and the silver nanoparticles tend to release active silver ions. These silver ions will attract and rapidly bind with the thiol group of the enzymes in bacteria, causing the thiol group-containing enzymes to lose their activity and the bacteria to die as a result. In particular, when the silver ions with positive charge are in contact with microorganism cells with negative charge, the former will be attracted to, and puncture the walls of, the latter, thus denaturing the microorganisms, compromising their ability to grow and preventing their metabolism and proliferation until they die. Silver nanoparticles, therefore, are highly effective in warding off and eliminating germs. Furthermore, silver ions will be released from the bacteria they kill, and repeat the foregoing process on other bacteria until all the bacteria accessible to them are eliminated. It can be known from the above that silver nanoparticles are advantageous in that they have long-term anti-pathogenic bacteria activity, do not cause drug resistance, are non-toxic and non-irritating, need not be activated by exposure to light and are not affected by pH values. Moreover, silver nanoparticles can inhibit the growth of mold and are antiseptic. Not only that, once silver is rendered into nanoparticles, the silver atoms exposed on the silver nanoparticle surface will increase rapidly, and that the silver nanoparticles in a solution will release silver ions continuously. The silver ions not only are the main contributor to the antibacterial effect, but also are incessantly released in the solution to make the antibacterial effect last. This explains why silver nanoparticles are a “permanent bactericide”.

Gold is an extremely stable and antioxidant material. Hence, apart from decorative applications, gold is used in Japan as a nourishing food ingredient and is added into tea, alcoholic drinks and various other ingredients. In the U.S. and Europe, gold is also used in cosmetics for wrinkle reduction and skin whitening and beautification. Clinically, gold is a common material for dental prostheses and coronary stents due to its excellent biocompatibility. While gold is highly stable, gold nanoparticles have unique electric potential properties that make the nanoparticles exceedingly adsorptive to oxygen and hence suitable for use as a catalyst. According to the findings of some Japanese scientists, gold nanoparticles are a catalyst capable of converting carbon monoxide into carbon dioxide through oxidation and therefore can be used to make surgical masks, gas masks, water heaters and so on to prevent carbon monoxide poisoning effectively. In contrast to industrial platinum catalysts, which will not react with the reactants unless at a high temperature and in the absence of water vapor, gold nanoparticle catalysts can react with the reactants at room temperature, whether there is water vapor or not. In addition, studies have shown that gold nanoparticles are highly effective (80 times as effective as vitamin C) in neutralizing free radicals. So, gold nanoparticles can also be added into biomedical materials to increase the in vivo stability, oxygen content and oxygen permeability of those materials.

Platinum has distinctive functionality and unique electric potential properties when rendered into nanoparticles of an average particle size of 2˜3 nm and evenly dispersed in a colloidal solution. The unique electric potential properties of platinum nanoparticles can be used to enhance the ability of the human skin to absorb water molecules and retain water for a long time, and this moisturizing effect lasts as long as the platinum nanoparticles exist in the cuticle of the skin. Furthermore, platinum nanoparticles will not be absorbed into and stored in the human body, and the colloid particles in a platinum nanoparticle-containing colloidal solution will have unique charge characteristics because of the platinum nanoparticles and therefore can absorb, and end up surrounded by, water molecules. When such colloid particles are attached to the human skin, not only will the water-retaining structure of the skin be instantly restored to its normal condition, but also the colloid particles will prevent the skin covered thereby from oxidation and enable the skin to maintain the water-retaining structure properly, so as to prevent the skin from being damaged in a dry environment, and to achieve the ultimate goal of improving the texture of the skin. It should be pointed out that platinum nanoparticles have generally the same negative electric potential as the human body when fixed in colloid particles, and that ionic balance is important to water retention in the skin. A platinum nanoparticle-containing colloid also has outstanding anion generation ability (more than about 16 times as efficient as forest bathing) and therefore can be used to balance the ions on the skin. Moreover, as platinum nanoparticles enclosed in colloid particles are kept from oxidation, they can generate anions semipermanently. In addition, the single electrons of the platinum nanoparticles will stay on the cuticle of the skin to which the nanoparticles are applied, and therefore are ready to catch free radicals swiftly; thus, platinum nanoparticles can also neutralize free radicals and are highly effective in inhibiting oxidation.

Palladium nanoparticles are an excellent catalyst and can rapidly absorb the hydrogen in water in a large amount. When palladium nanoparticles are used in conjunction with gold nanoparticles, the hydrogen absorbed by the palladium nanoparticles from water will gradually combine with the oxygen absorbed by the gold nanoparticles to form a hydrogen peroxide-like structure that can decompose the cell pigments in the epidermis of the user's skin without injuring the skin; as a result, a long-lasting spot fading effect is achieved.

The photocatalytic effects of the foregoing noble metal nanoparticles are briefly stated as follows, with particular reference to platinum, gold and palladium by way of example. When platinum, gold and palladium are rendered into nanoparticles, their crystal sizes are equivalent to or smaller than the wavelengths of light. This size effect increases the gap between their respective conduction bands and valence bands and thereby enhances the light absorbing ability of those noble metals. Therefore, when the noble metal nanoparticles are exposed to light, and a photon with a certain amount of energy, or energy exceeding the semiconductor band-gap energy, strikes the semiconductor electron structure of a platinum or gold nanoparticle, one electron in the valence band will be excited to the conduction band, leaving a hole behind. The hole in the valence band will capture the unpaired electron of a hydroxyl group in the surroundings and thus turn the hydroxyl group into a free radical. As such, the noble metal nanoparticles serve as a strong oxidant and can degrade organic substances such as pathogenic bacteria and viruses and eventually kill them.

Generally speaking, ions are electrically charged atoms, atomic groups or molecules, all of which are minute particles. Ions can be divided into “cations” (i.e., positively charged ions) and “anions” (i.e., negatively charged ions). Take water (H₂O) for example. The hydrogen (H) atom includes a positively charged proton orbited by a negatively charged electron. If the electron is driven away by an external force, the hydrogen atom will be positively charged and become a hydrogen ion (H⁺). The oxygen (O) atom, on the other hand, includes eight positively charged protons in its nucleus and eight electrons orbiting the nucleus, with two electrons in the inner orbital and six in the outer orbital. Since the outer orbital can accommodate a total of eight electrons and now has two vacancies, the oxygen atom is in a state in which it tends to capture electrons from the outside, the electron capturing process being referred to as “oxidation”. Hence, an oxygen atom, which is eager to capture electrons, will easily combine with two hydrogen atoms, each of which has an unpaired electron, to form a water molecule. When decomposed by the application of external energy, however, a water molecule breaks down into a positively charged hydrogen ion (H⁺) and a negatively charged hydroxyl ion (OH⁻). In a natural environment, hydroxyl ions exist as anions attached to water (H₂O+OH⁻=H₃O₂⁻). Given that water molecules are one of the most easily ionizable molecules in nature, the presence of a relatively large amount of anions in a certain environment indicates that anionic water molecules abound in the environment; in other words, the air in the environment contains a relatively large amount of anionic water molecules. Such an environment is generally considered to be conducive to a comfortable sensation, from which it follows that “anions” are also referred to as “comfortable ions”, “vitality ions” or “vitamin in the air”.

In light of the material properties of the aforesaid noble metal nanoparticles, namely the “highly efficient moisturizing effect”, the “highly efficient antibacterial effect” and the “long-term spot fading effect” of the silver, gold, platinum and palladium nanoparticles, the inventor of the present invention came up with the idea of adding those nanoparticles into the ingredients used to make cosmetic and skin care products, in order for the resulting composition to have a “highly efficient moisturizing effect”, a “highly efficient antibacterial effect” and a “long-term spot fading effect” and thereby compensate for the inadequacies of the conventional cosmetic and skin care products in terms of their “antibacterial”, “skin brightening and whitening” and “spot fading” effects. The nanoparticle-containing composition according to the present disclosure not only is suitable for use in the dry environment generated by an urban air conditioning system and allows a user's skin to maintain a comfortable and natural hydrated feel, but also produces a “highly efficient antibacterial effect” whereby the bacteria, viruses, mold or other organic substances in airborne impurities, dirt and dust will be degraded when such airborne pollutants are attached to the nanoparticle-containing composition. Thus, the “highly efficient antibacterial effect”, the “spot fading effect” and the “skin brightening and whitening effect” of the nanoparticle-containing composition can ward off and eliminate germs, ensure the health and safety of the user's skin and reduce the formation of pimple scars, spots or other blemishes on the skin should the user fail to clean the skin thoroughly.

The present disclosure also takes advantage of the “comprehensive UV blockage” property of zinc oxide nanoparticles and incorporates such nanoparticles into the preparation method of the nanoparticle-containing composition disclosed herein for highly efficient skin brightening, whitening, moisturizing and spot fading. Once the nanoparticle-containing composition is evenly applied over the user's skin, all of the aforesaid ingredients, including the zinc oxide nanoparticles, are evenly distributed over and attached to the skin, forming a composite protective film. The zinc oxide nanoparticles in the composite protective film not only can provide the skin with comprehensive UV protection, but also can be triggered by UV photons or other radiations (e.g., α rays, β rays, γ rays or X rays) to release electrons into the air through electron attachment or detachment, thereby converting the molecules in the air into anions carrying negative charges. The UV photons or other radiations will excite the noble metal nanoparticles as well as the zinc oxide nanoparticles such that the nanoparticles produce their respective photocatalytic effects. In other words, all the nanoparticles in the composite protective film will join forces to effectively degrade the bacteria, viruses, mold or other organic substances in contact with the film, thereby producing an exceptional antibacterial and sterilizing effect that makes the skin healthier and better-looking.

One embodiment according to the present disclosure provides a nanoparticle-containing composition for highly efficient skin brightening, whitening, moisturizing and spot fading. The nanoparticle-containing composition is prepared by mixing platinum nanoparticles, gold nanoparticles, palladium nanoparticles, silver nanoparticles, zinc oxide nanoparticles, tranexamic acid powder, a dispersant, deionized water and gelatin or a cream in predetermined weight percentages. The platinum nanoparticles constitute 5 ppm˜30 ppm by weight of the nanoparticle-containing composition, are evenly dispersed in the nanoparticle-containing composition, and can neutralize free radicals, help a user's skin to absorb moisture to raise a water content of the skin, and keep the skin delicate, supple and radiant. The gold nanoparticles constitute 3 ppm˜15 ppm by weight of the nanoparticle-containing composition; are evenly dispersed in the nanoparticle-containing composition; can stimulate the proliferation of, and the autosynthesis of collagen by, fibroblasts to invigorate the skin; and due to their outstanding antioxidation ability, can facilitate skin absorption the other beneficial ingredients of the nanoparticle-containing composition so as to lighten the spots or other blemishes on the skin surface. The palladium nanoparticles constitute 8 ppm˜32 ppm by weight of the nanoparticle-containing composition, are evenly dispersed in the nanoparticle-containing composition, are an excellent catalyst capable of rapidly absorbing the hydrogen in water in a large amount and can work with the gold nanoparticles such that the hydrogen absorbed by the palladium nanoparticles and the oxygen absorbed by the gold nanoparticles gradually combine into a hydrogen peroxide-like structure that can decompose the cell pigments in the epidermis of the skin and thereby lighten the spots on the skin persistently without injuring the skin. The silver nanoparticles constitute 120 ppm˜480 ppm by weight of the nanoparticle-containing composition, have particle sizes smaller than 10 nm, are evenly dispersed in the nanoparticle-containing composition, are totally harmless to the human body, and can effectively inhibit more than 600 species or strains of pathogenic bacteria, thereby preventing skin infection by those pathogenic bacteria and ensuring skin health effectively. The zinc oxide nanoparticles constitute 200 ppm˜1000 ppm by weight of the nanoparticle-containing composition, are evenly dispersed in the nanoparticle-containing composition, can provide comprehensive skin protection by reflecting and scattering UV radiation and can effectively prevent the skin from aging and having a dark and dull color after exposure to UV radiation. The tranexamic acid constitutes 1%˜5% by weight of the nanoparticle-containing composition. Tranexamic acid is water-soluble white crystalline powder and is odorless but slightly bitter in taste. The initial clinical use of tranexamic acid is to serve as an “antihemorrhagic agent” or “anticoagulant”. When a wound caused by a surgical procedure or trauma keeps bleeding, tranexamic acid is generally used to stop the blood flow. Intensive studies on the skin whitening and beautifying effect of tranexamic acid began only after a surgeon published his inadvertent clinical finding that tranexamic acid whitened the skin around the wound to which it was applied to stop blood flow, and the finding started the application of tranexamic acid to skin whitening and beautification. Recently, “tranexamic acid” was officially approved by the Ministry of Health and Welfare of Taiwan as a skin whitening and beautifying ingredient and has since become a popular additive widely recommended by major cosmetics brands for its skin whitening and beautifying effect. Tranexamic acid has been added to not only cosmetic and skin care products for topical use but also skin whitening and beautifying injections as a skin whitening and beautifying ingredient or additive. The deionized water constitutes 70%˜80% by weight of the nanoparticle-containing composition and is used to blend the aforesaid ingredients together. The dispersant constitutes 1%˜5% by weight of the nanoparticle-containing composition and is used in order for the aforesaid ingredients, except for the deionized water, to be evenly mixed and dispersed in the deionized water. The gelatin or cream serves as the gelation agent in the nanoparticle-containing composition so that the foregoing ingredients can be evenly mixed and dispersed in the nanoparticle-containing composition and be evenly applied over and adhere to the user's skin in order to produce long-lasting health-maintaining and cosmetic effects, including antibacterial, skin brightening, whitening, moisturizing and spot fading effects.

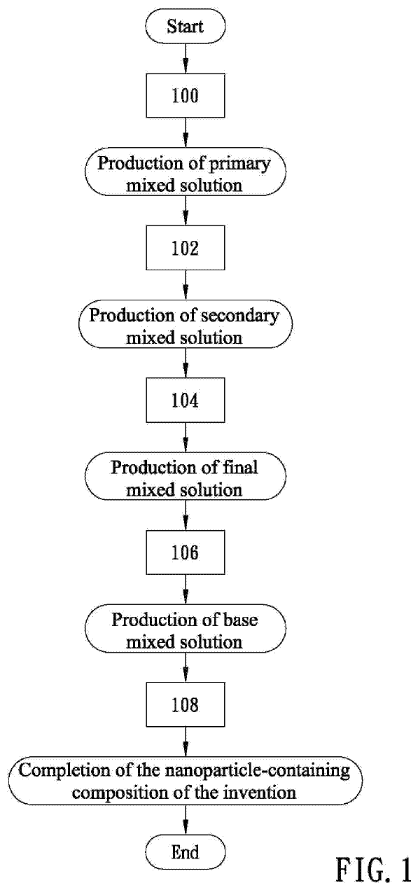

Another aspect of the present disclosure is directed to a preparation method of the aforesaid nanoparticle-containing composition for highly efficient skin brightening, whitening, moisturizing and spot fading. Referring to FIG. 1, the preparation method includes the following steps:

(100) An aqueous solution of the noble metal nanoparticles, including the platinum nanoparticles, the gold nanoparticles, the palladium nanoparticles and the silver nanoparticles, is mixed with an aqueous solution of the zinc oxide nanoparticles, and the mixture is stirred for at least 1 hour to produce a primary mixed solution in which the noble metal nanoparticles and the zinc oxide nanoparticles in the original aqueous solutions are sufficiently and evenly mixed and dispersed.

(102) The primary mixed solution is allowed to rest for at least 12 hours. After that, the clear upper-layer liquid of the primary mixed solution is extracted. The extracted clear upper-layer liquid is added with the deionized water to produce a secondary mixed solution.

(104) The secondary mixed solution is added with the tranexamic acid powder and the dispersant to produce a final mixed solution.

(106) The final mixed solution is heated to 40˜60° C. and stirred for at least 1 hour until the tranexamic acid powder is dissolved and is evenly mixed and dispersed in the final mixed solution. Then, the final mixed solution is cooled down to room temperature (e.g., 25° C.), and the clear upper-layer liquid of the cooled final mixed solution is extracted as the base mixed solution of the nanoparticle-containing composition.

(108) The base mixed solution is sequentially added with a fragrance and the gelatin (or cream) and is heated to 40˜60° C. and stirred for at least 1 hour. The fragrance constitutes 0.1%˜1% by weight of the nanoparticle-containing composition, and the gelatin (or cream) constitutes 0.5%˜10% by weight of the nanoparticle-containing composition. Once the base mixed solution, the fragrance and the gelatin (or cream) are sufficiently and evenly mixed and have the desired gel-like (or cream-like) consistency, the nanoparticle-containing composition for highly efficient skin brightening, whitening, moisturizing and spot fading is completed. It should be pointed out that the timing of adding the fragrance is not limited to step (108); the fragrance can be added during any one of the step of producing the secondary mixed solution, the step of producing the final mixed solution, and the step of producing the base mixed solution. The fragrance may also be dispensed with, depending on product requirements.

The nanoparticle-containing composition can be easily and evenly applied over and adhere to the user's skin to form a composite protective film on the skin. The zinc oxide nanoparticles in the composite protective film not only can provide the skin with comprehensive UV protection, but also can be triggered by UV photons or other radiations (e.g., α rays, β rays, γ rays or X rays) to release electrons into the air through electron attachment or detachment, thereby converting the molecules in the air into anions, which are negatively charged. The UV photons or other radiations will excite the noble metal nanoparticles as well as the zinc oxide nanoparticles such that the nanoparticles produce their respective photocatalytic effects. That is to say, all the nanoparticles in the composite protective film will join forces to effectively degrade the bacteria, viruses, mold or other organic substances in contact with the film, thereby producing an exceptional antibacterial and sterilizing effect that makes the skin healthier. In addition, the zinc oxide nanoparticles will provide “comprehensive UV protection” for the skin, the gold nanoparticles will invigorate the skin and facilitate skin absorption and utilize the skin whitening and beautifying constituents of the tranexamic acid, and the palladium nanoparticles will work with the gold nanoparticles to produce a hydrogen peroxide-like structure that can decompose the cell pigments in the epidermis of the skin without injuring the skin. Thus, the platinum nanoparticles, the gold nanoparticles, the palladium nanoparticles, the silver nanoparticles, the zinc oxide nanoparticles and the tranexamic acid produce their respective long-lasting health-maintaining and/or cosmetic effects, including antibacterial, skin brightening, whitening, moisturizing and spot fading effects, on the skin.

It should be pointed out that the embodiments described above are only some preferred ones according to the present disclosure. Implementation thereof is by no means limited to those embodiments, and the foregoing preparation steps and conditions can be adjusted according to practical needs. For example, the time point at which a particular ingredient is added may be postponed or preponed, and the duration of a particular preparation condition may be increased or decreased, provided that the weight percentages of the ingredients of the resulting nanoparticle-containing composition for highly efficient skin brightening, whitening, moisturizing and spot fading fall with their respective ranges as stated herein. All such changes and modifications do not depart from the technical principle according to the present disclosure and shall be viewed as encompassed by the appended claims.

While the invention herein disclosed has been described by means of specific embodiments, numerous modifications and variations could be made thereto by those skilled in the art without departing from the scope of the invention set forth in the claims.

What is claimed is:

1. A nanoparticle-containing composition for highly efficient skin brightening, whitening, moisturizing and spot fading, comprising:
platinum nanoparticles evenly dispersed in an aqueous solution, wherein the platinum nanoparticles serve to neutralize free radicals and help a user's skin to absorb moisture to raise a water content of the user's skin, and wherein the platinum nanoparticles constitute 5 ppm˜30 ppm by weight of the nanoparticle-containing composition;
gold nanoparticles evenly dispersed in the aqueous solution, wherein the gold nanoparticles serve to invigorate the skin by stimulating proliferation of, and autosynthesis of collagen by, fibroblasts, facilitate skin absorption of skin brightening, whitening and spot fading ingredients of the nanoparticle-containing composition through antioxidation ability of the gold nanoparticles, and wherein the gold nanoparticles constitute 3 ppm˜15 ppm by weight of the nanoparticle-containing composition;
palladium nanoparticles evenly dispersed in the aqueous solution, wherein the palladium nanoparticles are an excellent catalyst, are capable of rapid absorption of hydrogen in water in a large amount, and are able to work with the gold nanoparticles such that the hydrogen absorbed by the palladium nanoparticles and oxygen absorbed by the gold nanoparticles gradually combine into a hydrogen peroxide-like structure for decomposing a cell pigment in an epidermis of the skin without injuring the skin, and wherein the palladium nanoparticles constitute 8 ppm˜32 ppm by weight of the nanoparticle-containing composition;
silver nanoparticles evenly dispersed in the aqueous solution, wherein the silver nanoparticles have particle sizes smaller than 10 nm and constitute 120 ppm˜480 ppm by weight of the nanoparticle-containing composition;
zinc oxide nanoparticles evenly dispersed in the aqueous solution, wherein the zinc oxide nanoparticles serve to provide comprehensive skin protection by reflecting and scattering ultraviolet radiation, and wherein the zinc oxide nanoparticles constitute 200 ppm˜1000 ppm by weight of the nanoparticle-containing composition;
tranexamic acid constituting 1%˜5% by weight of the nanoparticle-containing composition, wherein the tranexamic acid is water-soluble white crystalline powder and serves to whiten and beautify the skin;
deionized water for blending the platinum nanoparticles, the gold nanoparticles, the palladium nanoparticles, the silver nanoparticles, the zinc oxide nanoparticles and the tranexamic acid together, wherein the deionized water constitutes 70%˜80% by weight of the nanoparticle-containing composition;
a dispersant allowing the platinum nanoparticles, the gold nanoparticles, the palladium nanoparticles, the silver nanoparticles, the zinc oxide nanoparticles and the tranexamic acid to be evenly mixed and dispersed in the deionized water, wherein the dispersant constitutes 1%˜5% by weight of the nanoparticle-containing composition; and
a gelation agent constituting 0.5%˜10% by weight of the nanoparticle-containing composition, wherein the gelation agent allows the platinum nanoparticles, the gold nanoparticles, the palladium nanoparticles, the silver nanoparticles, the zinc oxide nanoparticles, the tranexamic acid and the dispersant to be evenly mixed and dispersed in the nanoparticle-containing composition, thereby allowing the nanoparticle-containing composition to be evenly applied over and adhere to the skin.

2. The nanoparticle-containing composition of claim 1, wherein the gelation agent is gelatin, and the gelatin constitutes 0.5%˜3% by weight of the nanoparticle-containing composition.

3. The nanoparticle-containing composition of claim 1, wherein the gelation agent is a cream, and the cream constitutes 5˜10% by weight of the nanoparticle-containing composition.

4. The nanoparticle-containing composition of claim 1, further comprising a fragrance constituting 0.1˜1% by weight of the nanoparticle-containing composition.

5. The nanoparticle-containing composition of claim 2, further comprising a fragrance constituting 0.1˜1% by weight of the nanoparticle-containing composition.

6. The nanoparticle-containing composition of claim 3, further comprising a fragrance constituting 0.1˜1% by weight of the nanoparticle-containing composition.

7. A preparation method of a nanoparticle-containing composition for highly efficient skin brightening, whitening, moisturizing and spot fading, the preparation method comprising the steps of:
mixing an aqueous solution of noble metal nanoparticles with an aqueous solution of zinc oxide nanoparticles to form a resulting mixture, and stirring the resulting mixture for at least 1 hour to produce a primary mixed solution in which the noble metal nanoparticles and the zinc oxide nanoparticles in the aqueous solutions are sufficiently and evenly mixed and dispersed;
resting the primary mixed solution for at least 12 hours, extracting a first clear upper-layer liquid of the primary mixed solution, and adding deionized water into the extracted first clear upper-layer liquid to produce a secondary mixed solution;
adding tranexamic acid powder and a dispersant into the secondary mixed solution to produce a final mixed solution;
heating the final mixed solution to 40˜60° C. and stirring the final mixed solution for at least 1 hour until the tranexamic acid powder is dissolved and is evenly mixed and dispersed in the final mixed solution, cooling the final mixed solution down to room temperature, and extracting a second clear upper-layer liquid of the cooled final mixed solution as a base mixed solution of the nanoparticle-containing composition; and
adding gelatin or a cream into the base mixed solution, heating the base mixed solution to 40˜60° C., and stirring the base mixed solution for at least 1 hour such that the base mixed solution and the gelatin or the cream are sufficiently and evenly mixed and have a desired consistency, thereby completing the nanoparticle-containing composition for highly efficient skin brightening, whitening, moisturizing and spot fading, wherein the gelatin or the cream constitutes 0.5%˜10% by weight of the nanoparticle-containing composition.

8. The preparation method of claim 7, wherein the aqueous solution of the noble metal nanoparticles constitutes 70%˜80% by weight of the nanoparticle-containing composition; the noble metal nanoparticles comprise platinum nanoparticles, gold nanoparticles, palladium nanoparticles, and silver nanoparticles; the platinum nanoparticles constitute 5 ppm˜30 ppm by weight of the nanoparticle-containing composition; the gold nanoparticles constitute 3 ppm˜15 ppm by weight of the nanoparticle-containing composition; the palladium nanoparticles constitute 8 ppm˜32 ppm by weight of the nanoparticle-containing composition; and the silver nanoparticles constitute 120 ppm˜480 ppm by weight of the nanoparticle-containing composition.

9. The preparation method of claim 7, wherein the aqueous solution of the noble metal nanoparticles constitutes 70%˜80% by weight of the nanoparticle-containing composition; the noble metal nanoparticles comprise platinum nanoparticles, gold nanoparticles, and palladium nanoparticles; the platinum nanoparticles constitute 5 ppm-30 ppm by weight of the nanoparticle-containing composition; the gold nanoparticles constitute 3 ppm˜15 ppm by weight of the nanoparticle-containing composition; and the palladium nanoparticles constitute 8 ppm˜32 ppm by weight of the nanoparticle-containing composition.

10. The preparation method of claim 7, wherein the tranexamic acid powder constitutes 1%˜5% by weight of the nanoparticle-containing composition.

11. The preparation method of claim 8, wherein the tranexamic acid powder constitutes 1%˜5% by weight of the nanoparticle-containing composition.

12. The preparation method of claim 9, wherein the tranexamic acid powder constitutes 1%˜5% by weight of the nanoparticle-containing composition.

13. The preparation method of claim 7, wherein the dispersant constitutes 1%˜5% by weight of the nanoparticle-containing composition.

14. The preparation method of claim 8, wherein the dispersant constitutes 1%˜5% by weight of the nanoparticle-containing composition.

15. The preparation method of claim 9, wherein the dispersant constitutes 1%˜5% by weight of the nanoparticle-containing composition.

16. The preparation method of claim 7, wherein the gelatin, serving as a gelation agent, constitutes 0.5%˜3% by weight of the nanoparticle-containing composition.

17. The preparation method of claim 8, wherein the gelatin, serving as a gelation agent, constitutes 0.5%˜3% by weight of the nanoparticle-containing composition.

18. The preparation method of claim 9, wherein the gelatin, serving as a gelation agent, constitutes 0.5%˜3% by weight of the nanoparticle-containing composition.

19. The preparation method of claim 7, wherein the cream, serving as a gelation agent, constitutes 5%˜10% by weight of the nanoparticle-containing composition.

20. The preparation method of claim 8, wherein the cream, serving as a gelation agent, constitutes 5˜10% by weight of the nanoparticle-containing composition.

21. The preparation method of claim 9, wherein the cream, serving as a gelation agent, constitutes 5˜10% by weight of the nanoparticle-containing composition.

22. The preparation method of claim 7, further comprising adding a fragrance into the secondary mixed solution during the step of producing the secondary mixed solution, into the final mixed solution during the step of producing the final mixed solution, or into the base mixed solution during the step of producing the base mixed solution, wherein the fragrance constitutes 0.1%˜1% by weight of the nanoparticle-containing composition.

23. The preparation method of claim 8, further comprising adding a fragrance into the secondary mixed solution during the step of producing the secondary mixed solution, into the final mixed solution during the step of producing the final mixed solution, or into the base mixed solution during the step of producing the base mixed solution, wherein the fragrance constitutes 0.1%˜1% by weight of the nanoparticle-containing composition.

24. The preparation method of claim 9, further comprising adding a fragrance into the secondary mixed solution during the step of producing the secondary mixed solution, into the final mixed solution during the step of producing the final mixed solution, or into the base mixed solution during the step of producing the base mixed solution, wherein the fragrance constitutes 0.1%˜1% by weight of the nanoparticle-containing composition.
